(19) United States
(12) Patent Application Publication
(10) Pub. No.: US 2006/0149216 A1
(43) Pub. Date: Jul. 6, 2006

(54) TREATING SLEEP DEPRIVATION USING ANESTHETIC AGENT

(76) Inventor: John Sherman, Summerton, SC (US)

Correspondence Address: TILLMAN WRIGHT, PLLC, PO BOX 471581, CHARLOTTE, NC, 28247, US

(21) Appl. No.: 11/164,811

(22) Filed: Dec. 6, 2005

Related U.S. Application Data
(60) Provisional application No. 60/633,659, filed on Dec. 6, 2004.

(51) Int. Cl. A61M 31/00; A62B 17/04

(52) U.S. Cl. 604/503; 128/201.23

(57) ABSTRACT

A method for treating a person having sleep deprivation includes actively inducing unconsciousness in a person by administering an anesthetic agent to the person; determining an indication of a stage of sleep of the unconscious person by monitoring brainwaves of the person; and maintaining, for a predetermined period of time, the person in a state of sleep indicated to be deep sleep by adjusting the amount of the anesthetic agent being administered to the person. Preferably, the anesthetic agent is administered for about seven hours at different rates, and no surgical or other medical operation is performed. The anesthetic agent preferably is administered for the sole purpose of treating sleep deprivation.

### CROSS-REFERENCE TO RELATED APPLICATION

The present application is a nonprovisional patent application of, and hereby claims priority to, U.S. provisional patent application Ser. No. 60/633,659, filed Dec. 6, 2004, which is hereby incorporated herein by reference.

### COPYRIGHT STATEMENT

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### FIELD OF THE INVENTION

The present invention relates to treating persons with sleep deprivation and, in particular, to treating persons with sleep deprivation using anesthesia.

### BACKGROUND OF THE INVENTION

A hypnogram is a graphic representation of a sleeping period, showing the timing, duration, and sequence of every sleep stage an individual has throughout each consecutive cycle. People generally experience several cycles of sleep stages during a single night of sleep.

There are five conventionally recognized stages of sleep: sleep stage 1, sleep stage 2, sleep stage 3, sleep stage 4, and REM (rapid eye movement) sleep. Each stage represents a different physical and mental state of unconsciousness of the body during sleep, and the body cycles through the different sleep stages from sleep stage 1 to REM sleep. During some stages, the body is in a lighter sleep and can be awakened more easily, while other stages of sleep represent a very deep sleep. In this respect, each stage of sleep as conventionally understood is described as follows:

Stage 1 (Drowsiness): During this stage, a person drifts in and out of sleep for about five to ten minutes and can be awakened easily. The person's eyes move very slowly and muscle activity slows.

Stage 2 (Light Sleep): During this stage, eye movements stop and brain waves slow, with occasional bursts of rapid waves called sleep spindles. The heart rate also slows and body temperature decreases.

Stages 3 and 4 (Deep Sleep): It is very difficult to wake someone during sleep stages 3 and 4, which are considered deep sleep. During sleep stage 3, slow brain waves called delta waves begin to appear, interspersed with smaller, faster brain waves. By sleep stage 4, the brain produces delta waves almost exclusively and generally no eye movement or muscle activity occurs. A person awakened during deep sleep does not adjust immediately and often feels groggy and disoriented for several minutes after awakening.

REM Sleep: During REM sleep, breathing becomes more rapid, irregular, and shallow, the eyes jerk rapidly in various directions, and the limb muscles become temporarily paralyzed. The heart rate also increases and blood pressure rises. Dreams also occur during this sleep stage.

Forty million Americans are believed to be chronically sleep deprived with an additional twenty million people experiencing occasional sleeping problems. Nationally, this has significant health and financial consequences. Chronic sleep deprivation impacts general wellness and many particular aspects of health including immune responses and susceptibilities to illnesses. It is thought to be related to disorders of the nervous and cardiovascular systems. Additionally, those who suffer chronic sleep problems are susceptible to decreased awareness and errors of judgment and thereby suffer needless injuries and deaths due to accidents that could be prevented. It is estimated that chronic sleep deprivation directly costs this country sixteen billion dollars annually, although the total impact of lost productivity and other indirect costs is probably much greater. Increasing demands are placed on the busy schedules of the people of industrialized nations while a general lack of awareness toward sleep deprivation is promoting an overlooked health crisis.

Historically, the medical system has focused on those individuals identified as having clinically diagnosed sleep disorders. This limited focus omits consideration toward intermittent or chronic sleep deprivation having temporary, situational, or habitual causes. Thus, those who suffer sleep deprivation are generally without any assistance in facing problems that have real medical consequences.

Many sedative medicines negatively impact sleep architecture. Nonrapid eye movement (NREM) sleep stages 3 and 4 are the sleep stages most responsible for restful restorative sleep. However, sedatives historically used to treat sleep disorders typically result in increased stage 2 sleep without promoting sleep in stages 3 and 4, thereby failing to promote restorative sleep continuity. For example, classic benzodiazepines (which are sedative hypnotics) are prescribed to treat sleep disorders though they cause decreases in slow-wave sleep duration, decreases in delta power during slow wave sleep, and increases in stage 2 NREM sleep.

### SUMMARY OF THE INVENTION

The present invention relates to treating persons having sleep deprivation. In this regard, technologies from the medical fields of anesthesiology and sleep disorder treatment-fields that have historically developed generally independently of each other—are incorporated into one or more preferred embodiments of the invention.

The present invention includes many aspects and features. According to one or more aspects of the invention, a medically designed patient program is used for directly treating sleep deprivation. A person with significant sleep deprivation with or without a sleep disorder is provided an opportunity to seek sleep education, counseling, and notably, active induced sleep restoration. Furthermore, one or more aspects of the invention relate to a practical outpatient application in a new field of “sleep anesthesia.”

In another aspect, the invention particularly relates to an acute phase sleep restoration program for inducing a patient or other person being treated to improve from a severe state of sleep deprivation to a safe state of sleep restoration. According to this aspect, a sufficiently high delta powered sleep is induced to allow physiologic recovery from a severe sleep deprivation state. This is achieved in part by utilizing an active process of sleep induction (as opposed to a passive process of natural sleep that sleep-deprivation sufferers attempt without success). The active process of sleep induction preferably is effected by way of a medicinal infusion titrated to induce an ordered sleep cycle within which the treated person's sleep is maintained at NREM sleep stages 3 and 4 for a predetermined period of time.

In a feature of the invention, the treatment is performed during the treated person's normal time for sleeping. Importantly, because the timing of the active process of sleep induction is selected and controlled, the circadian rhythms of the person are reinforced. (As is conventionally understood, the “circadian rhythms” represent the set of biological rhythms that are completed in a day and that regulate the behavioral and physiological processes to be more active during the person's normal “day time” and less active during the person's normal “night time”.

In other features of the invention, body homeostasis in the sleep environment is maintained as closely to natural sleep as possible and personal comfort at the time of awakening is promoted to minimize residual effects of the treatment.

An anesthetic agent that appears to closely mimic natural sleep is 2-6 diisopropylphenol (also called “2,6 diisopropyl phenol; “propofol”; “diprivan”; “diisopropylphenol”; “2,6-bis(1-methylethyl)phenol”; and “disoprofol”). It is believed that, prior to the inventive sleep restoration treatment of the invention, 2-6 diisopropylphenol has not been utilized to treat sleep deprivation. It also is believed that 2-6 diisopropylphenol acts by binding to gamma-aminobutyric acid, a receptor at a site distinct from the benzodiazepine binding site. The chemical formula for 2-6 diisopropylphenol is C₁₂H₁₈O.

Used in accordance with the invention, it is believed that 2-6 diisopropylphenol induces NREM stages of restful, restorative deep sleep for controlled periods of time. While no anesthetic agent equivalent to 2-6 diisopropylphenol is believed to exist, current and future research and development may provide anesthetic agents equivalent to equivalent that increasingly induce natural mechanisms of sleep, increasingly overlap neurobiologic mechanisms with natural sleep, and minimize pharmacologic imposition on the treated person's natural system. Thus, while 2-6 diisopropylphenol is discussed herein with regard to one or more preferred embodiments of the invention, the use of other currently available anesthetic agents are within the scope of each claimed invention unless otherwise limited explicitly by the language of the claim to 2-6 diisopropylphenol and future equivalents thereof.

In addition to the aforementioned aspects and features of the present invention, it should be noted that the present invention further includes the various possible combinations of such aspects and features. Examples of such combinations are illustrated in the detailed description set forth below and drawings referenced therein.

Referring now to the claims and with regard to a summary thereof, a method for treating a person having sleep deprivation in accordance with one aspect of the invention includes the steps of: actively inducing unconsciousness in a person by administering an anesthetic agent to the person; determining an indication of a stage of sleep of the unconscious person by monitoring brainwaves of the person; and maintaining, for a predetermined period of time, the person in a state of sleep indicated in said step (b) to be deep sleep by adjusting the amount of the anesthetic agent being administered to the person.

The administration of the anesthetic agent and the determination of an indication of a stage of sleep are at least intermittently performed during the predetermined period of time and, preferably, are continuously performed.

The predetermined period of time preferably is between approximately four hours and approximately six hours, and may be about five hours. Such a period of time generally is otherwise insufficient for many people to receive a good night's sleep, which now may be had as a benefit of the present invention.

The method also preferably is performed during the time for sleeping in accordance with the circadian rhythms of the person receiving the treatment.

In a feature of the invention, the administering of the anesthetic agent to the person is performed via an IV and an infusion pump.

In another feature, the method further includes awakening the person by ceasing the administration of the anesthetic agent when approximately the seventh hour of performance of the method has transpired.

In yet another feature, the method further includes the steps of monitoring blood pressure, cardiac rhythm, oxygen saturation, and body temperature of the person at least while the anesthetic agent is being administered.

In yet another feature, the method further includes the steps of maintaining the person in a reclined position during performance of the method such that hip and knee joints of the person are slightly flexed during performance of the method. The person preferably is maintained in a reclined position in a padded chair during performance of the method. The method also preferably includes the additional step of reducing risks of obstructive sleep apnea by administering oxygen to supplement the delivered air with positive airway pressure while the unconscious person is reclined.

In another feature, the method includes the step of administering supplemental oxygen to the person using a nasal cannula. Alternatively, the method includes the steps of administering oxygen to the person using a nasal cannula and, thereafter, administering oxygen to the person using a facemask, the nasal cannula being replaced by the facemask for accommodating breathing both through the nose and the mouth of the person while the person is in a sleep-like state.

In administering oxygen to the person using a facemask, the method also preferably includes the step of monitoring carbon dioxide in the exhaled air within the facemask. The monitoring of the carbon dioxide preferably is performed using a capnograph. In still yet further features, the method preferably includes the step of reducing body temperature decreases through respiratory heat losses by heating air that is delivered to the facemask for inhalation, and reducing mucosal swelling by humidifying air that is delivered to the facemask for inhalation.

Still yet another aspect of the invention relates to a breathing device for a person having sleep-disordered breathing. The breathing device comprises a titratable machine adapted to deliver heated and humidified air with bi-level positive airway pressure and adapted to introduce a small amount of supplemental oxygen into the delivered air, and a facemask for inhalation of the delivered air. In accordance with the invention, the breathing apparatus includes the improvement comprising a sampling tube attached to the facemask connected to a capnographer for monitoring carbon dioxide in exhaled air in the facemask. The breathing machine also preferably is auto-titratable.

In another aspect of the invention, a method for treating a person having sleep deprivation includes the steps of: actively inducing unconsciousness in a person by administering an anesthetic agent to a person; determining an indication of a stage of sleep of the unconscious person by monitoring brainwaves of the person; and maintaining, for a predetermined period of time, the person in a state of sleep indicated in said step (b) to be deep sleep by adjusting a rate at which the anesthetic agent is administered to the person. In accordance with this aspect, actively inducing the unconsciousness in the person includes the steps of administering the anesthetic agent at an initial rate such that the person initially experiences a state of sleep indicated to be sleep stages 1 and 2, and thereafter administering the anesthetic agent at a second rate approximately twice that of the initial rate such that the person experiences deep sleep. The initial rate preferably is gradually increased to the second rate such that the person transitions through what is indicated to be sleep stages 1 and 2 in reaching the deep sleep. In this regard, the initial rate preferably is gradually increased to the second rate over approximately one hour. Furthermore, the anesthetic agent preferably is administered at the second rate for approximately five hours, and the anesthetic agent preferably is administered at a third rate that is half of the second rate after approximately the sixth hour. The anesthetic agent is administered at the third rate for approximately one hour to allow for natural burst cycling events such as sleep spindles, k complexes, and REM sleep.

In still yet another aspect, a method for treating a person having sleep deprivation includes the steps of: actively inducing a sleep-like state in a person by administering an anesthetic agent to a person; determining an indication of a stage of sleep of the person by monitoring brainwaves of the person; maintaining, for a predetermined period of time, the person in a sleep-like state indicated in said step (b) to be deep sleep by adjusting the amount of the anesthetic agent being administered to the person; and minimizing residual effects of the anesthetic agent in the person upon awakening of the person. The residual effects are minimized by: administering an IV fluid bolus for eliminating accumulation of lactic acid in muscles of the person, such as a bolus of IV fluid comprising a saline solution; massaging the person for eliminating accumulation of lactic acid in muscles of the person, including performing a myofascial massage; and/or providing food to the person for consumption, such as a light breakfast.

In another aspect of the invention, a method for treating sleep deprivation in a person by actively inducing sleep in the person such that the person experiences predetermined stages of sleep for predetermined periods of time. This method includes the steps of administering an anesthetic agent to the person while monitoring brainwaves of the person, and adjusting the amount of anesthetic agent being administered to the person such that the person experiences the predetermined stages of sleep for the predetermined periods of time. The predetermined periods of time preferably are contiguous and add up to between about six hours and about eight hours and, preferably, about eight hours.

In accordance with yet another aspect of the invention, a commercial method of providing sleep restoration services to a person having a predefined window of time available in which to rest includes the step of actively inducing sleep by administering 2-6 diisopropylphenol to the person such that a predetermined stage of sleep is maintained for a predetermined period of time, said predetermined period of time not being greater than the predefined window of time. The predetermined stage of sleep is either sleep stage 3 and/or sleep stage 4, and the predetermined period of time is between about four hours and about six hours.

In a similar aspect of the invention, a commercial method of providing sleep restoration services to a person having a predefined window of time available in which to rest includes the steps of actively inducing sleep by administering 2-6 diisopropylphenol to the person such that predetermined stages of sleep are maintained for predetermined periods of time, the sum of said predetermined periods of time not being greater than the predefined window of time. A first predetermined stage of sleep is stage 1 and/or 2 and a second predetermined stage of sleep is sleep stages 3 and/or 4. A first predetermined period of time is about one hour and a second predetermined period of time is between about four hours and about six hours.

In still another aspect of the invention, a method of conducting a commercial business that provides sleep restoration services includes the steps of: advertising sleep restoration services, and in exchange for monetary consideration, providing sleep restoration services that include the steps of: (i) actively inducing sleep in a person such that the person experiences predetermined stages of sleep for predetermined periods of time by administering an anesthetic agent to the person while monitoring brainwaves of the person, and (ii) adjusting the amount of anesthetic agent being administered to the person such that the person experiences the predetermined stages of sleep for the predetermined periods of time.

In features of this business method, the sleep restoration services are advertised for and provided to commercial airline pilots; are advertised for and provided to commercial truck drivers; are advertised for and provided to commercial airline pilots; and/or are advertised for and provided to air traffic controllers.

Another aspect of the invention relates to a method of providing sleep restoration services to a member of the armed services (i.e., to a serviceperson) who has a predefined, limited window of time available in which to rest. The method includes the steps of actively inducing sleep by administering an anesthetic agent to the serviceperson such that a deep sleep is maintained for a predetermined period of time, the predetermined period of time not being greater than the predefined window of time. The predefined window of time comprises between about six hours and about eight hours prior to initiation of a mission by the serviceperson. The period of time also may not correspond to the time for sleeping in accordance with the circadian rhythms of the serviceperson, whereby this aspect of the present invention may be extremely useful. The serviceperson may comprise, for example, a soldier. The serviceperson also may comprise an aircraft pilot, and the predefined window of time may be between about six hours and eight hours prior to a flight mission of the pilot.

In one or more aspects of the invention, the anesthetic agent is administered for treatment to a person having sleep deprivation, or otherwise seeking to receive restful sleep, without any surgical or other medical procedure being performed upon the person while the anesthetic agent is administered.

Additionally, in one or more of the foregoing aspects, the anesthetic agent preferably is 2-6 diisopropylphenol or an equivalent anesthetic that is capable of producing a sleep-like state including sleep stages 3 and 4 (i.e., deep sleep) during which the body rejuvenates itself.

In still yet other aspects of the invention, chronic sleep deprivation is treated by addressing acute sleep deprivation as set forth in one or more aspects above in combination with providing cognitive behavioral therapy to the person over multiple sessions.

### BRIEF DESCRIPTION OF THE DRAWINGS

One or more preferred embodiments of the present invention now will be described in detail with reference to the accompanying drawings, wherein the same elements are referred to with the same reference numerals, and wherein:

FIG. 1 is a flowchart illustrating a general method in accordance with the present invention;

FIG. 2 is a flowchart illustrating an application of the general method of FIG. 1 in greater detail;

FIGS. 3, 4 and 5 collectively represent a detailed flowchart illustrating a preferred method of the present invention;

FIG. 6 is a graphical illustration of an anesthetic infusion rate used over the course of treatment of a person having sleep deprivation; and

FIG. 7 is a graphical illustration of an approximate level of consciousness of a subject during the time period of the treatment of FIG. 6.

### DETAILED DESCRIPTION

As a preliminary matter, it will readily be understood by one having ordinary skill in the relevant art (“Ordinary Artisan”) that the present invention has broad utility and application. Furthermore, any embodiment discussed and identified as being “preferred” is considered to be part of a best mode contemplated for carrying out the present invention. Other embodiments also may be discussed for additional illustrative purposes in providing a full and enabling disclosure of the present invention. Moreover, many embodiments, such as adaptations, variations, modifications, and equivalent arrangements, will be implicitly disclosed by the embodiments described herein and fall within the scope of the present invention.

Accordingly, while the present invention is described herein in detail in relation to one or more embodiments, it is to be understood that this disclosure is illustrative and exemplary of the present invention, and is made merely for the purposes of providing a full and enabling disclosure of the present invention. The detailed disclosure herein of one or more embodiments is not intended, nor is to be construed, to limit the scope of patent protection afforded the present invention, which scope is to be defined by the claims and the equivalents thereof. It is not intended that the scope of patent protection afforded the present invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection afforded the present invention is to be defined by the appended claims rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term-differs in any way from any particular dictionary definition of such term, it is intended that the meaning of the term as understood by the Ordinary Artisan should prevail.

Furthermore, it is important to note that, as used herein, “a” and “an” each generally denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at lease one of the items,” but does not exclude a plurality of items of the list. Thus, reference to “a picnic basket having cheese or crackers” describes “a picnic basket having cheese without crackers”, “a picnic basket having crackers without cheese”, and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

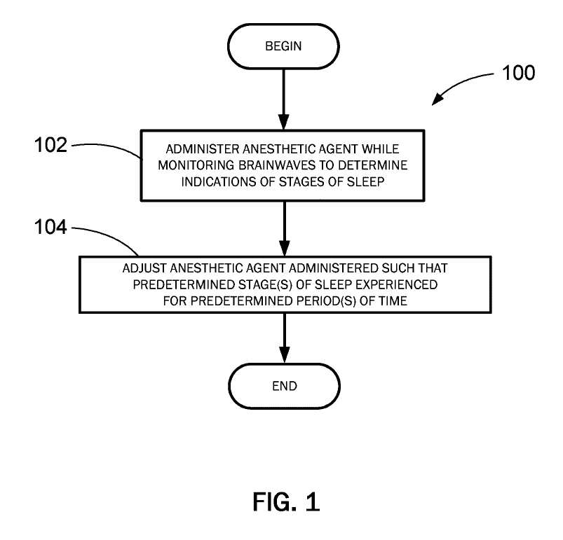

Turning now to the drawings, a preferred embodiment 100 of the invention for treating a person with sleep deprivation is illustrated in FIG. 1. As shown therein, the present invention comprises a method of treating sleep deprivation using an anesthetic agent. In a first step 102, the anesthetic agent is administered to the sleep-deprived person while the person's brainwaves are monitored so that an indication of the person's various stages of sleep may be determined. In a second step 104, the anesthetic agent being administered is adjusted such that the person experiences one or more predetermined stages of sleep for a predetermined period or periods of time.

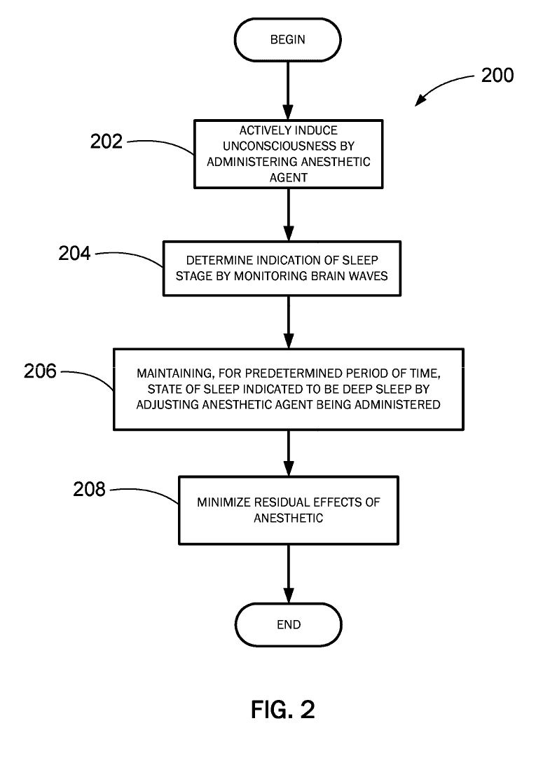

FIG. 2 is a simplified flowchart illustrating an application 200 of the general method 100 of FIG. 1 in greater detail. As shown in FIG. 2, unconsciousness is actively induced in the sleep-deprived person at step 202 by administering the anesthetic agent. At step 204, an indication of the person's sleep stage is determined by monitoring the person's brain waves. Next, at step 206, a state of sleep indicated to be “deep sleep” is maintained for a predetermined period of time by adjusting the anesthetic agent being administered. Finally, at step 208, residual effects of the anesthetic are minimized.

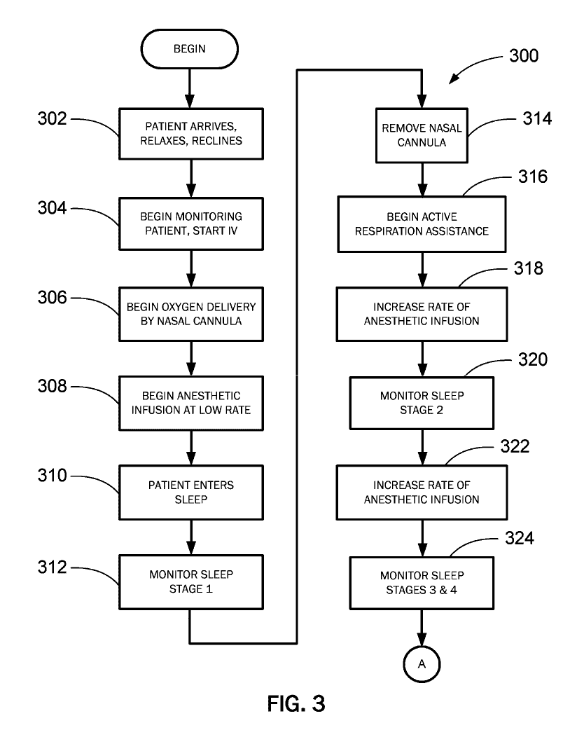

FIGS. 3, 4 and 5 collectively represent a detailed flowchart illustrating a preferred method 300 of the present invention. Although not shown therein, physical facilities for implementing methods of the present invention, such as preferred method 300, are first established. Such facilities may be include pre-existing medical facilities and treatment centers such as hospitals and the like, but many other types of facilities, separate from traditional medical facilities, are also envisioned, such as stand-alone sleep treatment centers, retail outlets in shopping centers, portable equipment for use by military personnel in the field or by other types of personnel, rest centers in airports or truck stops, and a wide variety of other implementations.

Regardless of the type of facility being used, the treatment protocol generally begins with the arrival of a person to be treated at the facility. The person may be a medical patient, a customer, a soldier or other serviceperson, an airplane pilot, a truck driver, or any other type of individual for whom sleep treatment may be desirable, required, prescribed or otherwise suitable. However, for the sake of clarity, the person being treated may be referred to hereinafter as a “person.” The person to be treated preferably arrives at the facility with suitable time prior to their normal sleep time for changing into comfortable clothing and completing any relaxing functions such as visiting the restroom if needed. The person may be previously advised to bring a book or magazine to read prior to and at the onset of the treatment. Preferably, the person's past medical history and other pertinent information will have been obtained prior to the visit to minimize administrative details from the perspective of the person to be treated. At step 302, once these preliminary matters have been addressed, the person being treated is asked to relax on, for example, a padded recliner such as a dental patient chair. Padding eliminates pressure points and the recliner helps prevent joint pain by keeping major joints such as hips and knees slightly flexed while the person sleeps motionlessly.

Once the person is positioned, an IV is set up at step 304 and the person is subjected to standard monitoring as set forth by guidelines from the American Society of Anesthesiologists for delivering anesthesia. Standard monitoring includes non-invasive blood pressure monitoring (blood pressure cuff), cardiac monitoring for rhythm, and use of pulse oximetry for blood oxygen saturation. Additionally, capnography may be utilized in conjunction with a breathing apparatus (as described in the following) to monitor for apnea and end tidal carbon dioxide level. A skin temperature sensor may be used to monitor trends in body temperature.

A commercially available monitor to record brain wave function is utilized to read a derived signal index from EEG lead points. Examples of available monitors to record brain wave function and/or indicate state of unconsciousness of a person include a bispectral index monitor (also known as a “BIS” monitor), a Narcotrend™ monitor, and a patient state analyzer. These instruments are designed for use in the field of anesthesiology to assess conscious level on a numerical scale from 100 (awake) to 0 and have not apparently been previously used in the field of sleep medicine. These types of monitors have been found to be reliable indicators of depth of sleep comparable to a full EEG montage and are discussed herein with relation to convenient and comfortable guidance of the titration of medicine to ensure target depth and duration of sleep.

The person's IV is connected to an adjustable infusion pump set to deliver 2-6 diisopropylphenol (propofol, diprivan). At step 306, the person being treated is given supplemental oxygen initially by nasal cannula. Because 2-6 diisopropylphenol is an anesthetic, it may be preferable or necessary for a trained and licensed anesthesia provider to administer the treatment.

At the onset of treatment, the person is fully monitored, given supplemental oxygen, and the titration is started. A goal of the medicine titration is to mimic the expected sleep architectural patterns of normal sleep. At sleep onset, a person typically passes through stages 1 and 2 followed by deeper stages of sleep 3 and 4. The titration is initiated at step 308 at approximately half the expected rate to achieve consistent slow wave sleep. Over time the rate of infusion for the treated individual is increased until the depth of sleep meets the stated goals. The slow initial titration promotes gradual passage through stages 1 and 2 and avoids over-shoot on the final infusion rate.

Early in the treatment process after the person has fallen asleep (step 310 in FIG. 3), the oxygen nasal cannula preferably is removed at step 314 and replaced by a breathing apparatus at step 316. This replacement process preferably takes place after it is determined, at step 312, that the person has entered sleep stage 1. The breathing apparatus is used as a non-invasive alternative to avoid the typical endotracheal intubation employed during general anesthesia. The preferred apparatus is a heated, humidified, bi-level positive airway pressure machine with a small amount of supplemental oxygen mixed into the delivered air, which machine is conventionally available from, for example, Respironics; is generally referred to as a BIPAP machine; and is conventionally used for sleep-disordered breathing. In accordance with an aspect of the invention, this machine is modified to include a sampling tube that is connected to the capnographer. Furthermore, the apparatus preferably is modified to be auto-titratable.

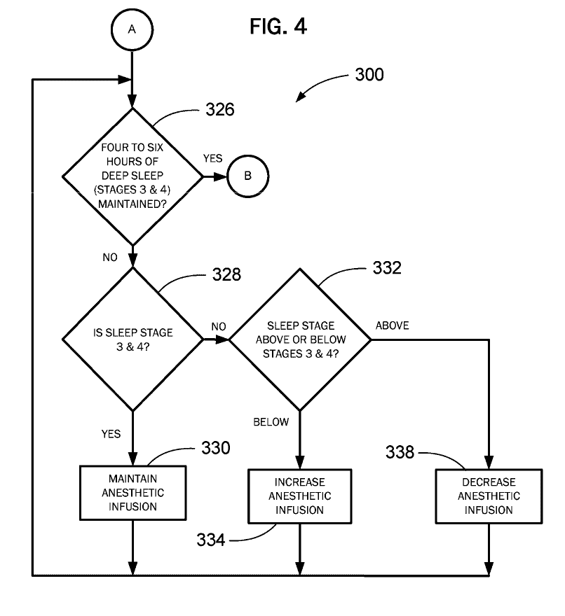

Gradually, the rate of anesthetic infusion is increased as shown at steps 318 and 322 in order to progress the person from sleep stage 1 to sleep stage 2, shown as being monitored at step 320, and finally into sleep stages 3 and 4, shown as being monitored at step 324. The general procedure for maintaining the person in deep sleep (stages 3 and 4) is illustrated in FIG. 4. The person is preferably maintained in deep sleep for between four and six hours, and more preferably for approximately five hours. If at step 326 this period of time has not yet passed, then the step of monitoring the person to determine the person's sleep stage is shown at step 328. If the person remains in sleep stage 3 or 4, then the anesthetic infusion remains constant at step 330, and steps 326, 328 and 330 are repeated. However, if the person is not in sleep stage 3 or 4, then at step 332 it is determined whether he is above or below stages 3 and 4. If below (e.g., at only sleep stage 2), then at step 334 the anesthetic infusion is increased, while if above, then the anesthetic infusion is decreased at step 338.

Utilizing the apparatus addresses various factors of the treatment. The positive airway pressure and recumbent position of the person in the recliner address difficulties with obstructive sleep apnea. A full-face mask configuration accommodates both nose and mouth breathing. Humidification minimizes mucosal swelling that might otherwise occur as caused by excessively dry air. Heated humidification avoids body temperature decreases through respiratory heat losses. Another consideration in deep sleep is the retention of carbon dioxide. This is addressed through the bi-level positive airway pressure feature of the apparatus. Adequate ventilation is assisted as the person initiates each breath and is monitored by a capnograph.

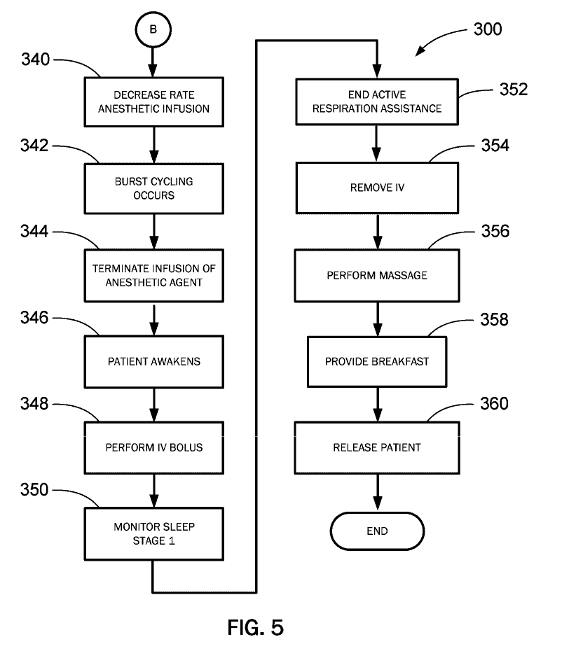

Referring now to FIG. 5, once the predetermined period of time has passed between the start of the infusion to the start of downward titration of the anesthetic agent (as determined at step 326 of the preferred treatment protocol), the titration is decreased at step 340 to about one-half the rate used for deep sleep and administered at that rate for approximately one hour to allow for natural burst cycling events, shown at step 342, such as sleep spindles, k complexes, and REM sleep. At step 344, the infusion is terminated at approximately the seventh hour. The medicine is metabolized and clears from the person's system gradually allowing the person to awaken from sleep at step 346.

When it is determined at step 350 that the person has awakened sufficiently, the mask and IV are removed at steps 352 and 354, respectively. Preferably, a bolus of IV fluid is given, at step 348, prior to the IV being removed. The person is optionally given a light breakfast and a deep myofascial massage at steps 358 and 356, respectively. The purpose of the IV fluid bolus and the massage therapy is to help eliminate any build up of lactic acid in the muscles that can occur with the use of 2-6 diisopropylphenol. When the person feels ready, he or she is released to go home at step 360. Additional sleep hygiene counseling and training are optionally arranged.

The concept of active sleep restoration for the general public and the process it entails is a novel approach. There are many reasons people do not get sufficient sleep. The inventive treatment promotes adequate duration of restorative deep sleep by applying anesthesia to address issues in sleep medicine. Exemplary medicines include 2-6 diisopropylphenol and upcoming anesthetic agents, the effects of which closely overlap neurobiological mechanisms of sleep.

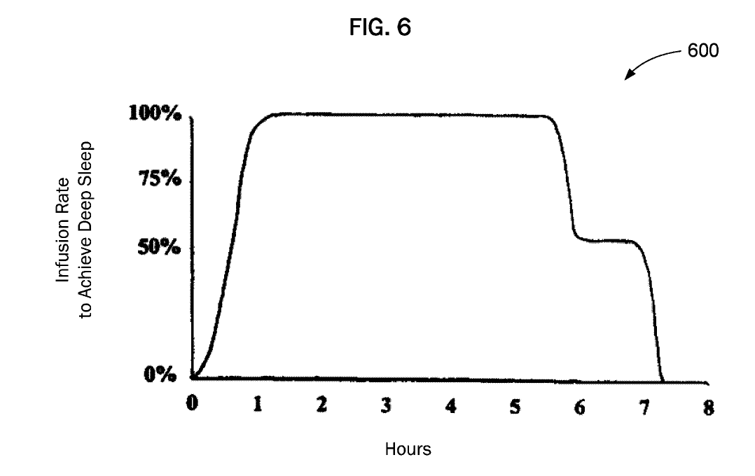

FIG. 6 is a graphical illustration 600 of the anesthetic infusion rate used during the period of treatment. As shown therein, the infusion rate is gradually increased from 0% to 100% during the first hour of treatment and then maintained generally constant for about five hours. After that, the rate is decreased by approximately 50% for an additional hour to allow for natural burst cycling events. Finally, the infusion rate is gradually reduced to zero to permit the person to awaken.

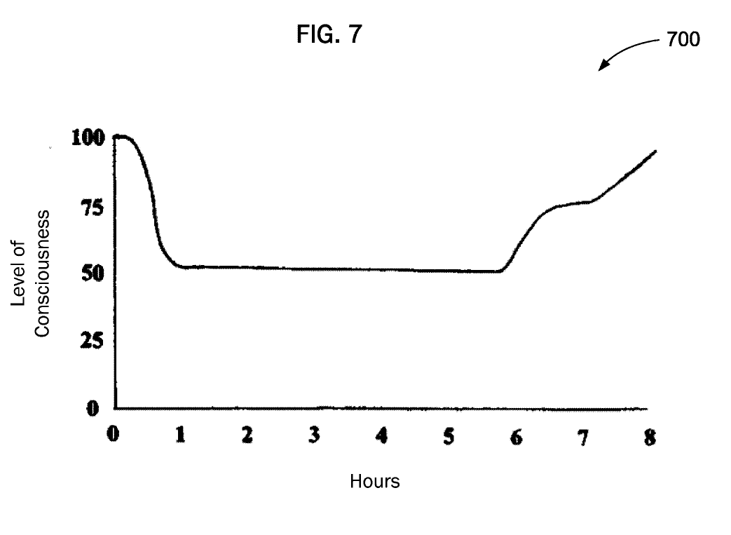

FIG. 7 is a graphical illustration 700 of the approximate level of consciousness of a subject during the treatment cycle of FIG. 6. As shown therein, the person is fully conscious at the beginning of treatment, but drops quickly to approximately 50% consciousness during the first hour of treatment, approximately in conjunction with the rate of anesthetic infusion being increased from 0% to 100% and averaging a rate of 50%. The person's state of consciousness remains relatively constant for about five hours as a full rate of anesthetic continues to be administered. The infusion rate is dropped to 50% at the sixth hour of administration of the anesthetic agent and then terminated at the seventh hour, after which the person gradually begins to awaken to a full state of consciousness.

In an implementation of the present invention, a commercial service for sleep restoration, and advertising therefor, is contemplated. In this regard, the preferred methods of the present invention may be utilized in providing the sleep restoration services to recipients such as airline pilots, air traffic controllers, and truck drivers, especially where the recipient does not have a routine schedule. Indeed, such commercial services would be of particular use to a person having a limited window of time for sleep, during which restorative sleep may not be practical or possible without the present invention. Similarly, the present invention may be used in military applications for providing restorative sleep to soldiers and other servicepersons, during a limited window of time that may be available. Such forced restorative sleep is particularly useful when the window of time that is available to the soldiers does not correspond well to the soldier's circadian rhythms.

Significantly, in many applications of the methods and processes of the present invention, it is believed that the steps thereof may be carried out by individuals who are not “medical practitioners” as that term is defined in the Patent Act. Likewise, in many implementations, it is believed that the steps themselves do not represent a “medical activity” as that term is defined in the Patent Act. Alternatively, depending upon the context and prevailing circumstances, other applications of the methods and processes of the present invention may be utilized by “medical practitioners” in a manner that constitutes a “medical activity.”

What is claimed is:

1. A method for treating a person having sleep deprivation, comprising the steps of:
(a) actively inducing unconsciousness in a person by administering an anesthetic agent to the person;
(b) determining an indication of a stage of sleep of the unconscious person by monitoring brainwaves of the person; and
(c) maintaining, for a predetermined period of time, the person in a state of sleep indicated in said step (b) to be deep sleep by adjusting the amount of the anesthetic agent being administered to the person.

2. The method of claim 1, wherein the anesthetic agent comprises 2-6 diisopropylphenol.

3. A method for treating a person having sleep deprivation, comprising the steps of:
(a) actively inducing unconsciousness in a person by administering an anesthetic agent to a person;
(b) determining an indication of a stage of sleep of the unconscious person by monitoring brainwaves of the person; and
(c) maintaining, for a predetermined period of time, the person in a state of sleep indicated in said step (b) to be deep sleep by adjusting a rate at which the anesthetic agent is administered to the person;
(d) wherein said step (a) comprises,
(i) administering the anesthetic agent at an initial rate such that the person initially experiences a state of sleep indicated in said step (b) to be sleep stages 1 and 2, and
(ii) thereafter, administering the anesthetic agent at a second rate approximately twice that of the initial rate such that the person experiences the deep sleep of said step (c).

4. The method of claim 3, wherein the anesthetic agent comprises 2-6 diisopropylphenol.

5. The method of claim 3, wherein the initial rate is gradually increased to the second rate such that the person transitions through what is indicated to be sleep stages 1 and 2 in reaching the deep sleep of said step (c).

6. The method of claim 3, wherein the initial rate is gradually increased to the second rate over approximately one hour.

7. The method of claim 3, wherein the anesthetic agent is administered at the second rate for approximately five hours.

8. The method of claim 3, wherein the anesthetic agent is administered at a third rate that is half of the second rate after approximately the sixth hour.

9. The method of claim 8, wherein the anesthetic agent is administered at the third rate for approximately one hour to allow for natural burst cycling events such as sleep spindles, k complexes, and REM sleep.

10. The method of claim 3, further comprising providing cognitive behavioral therapy to the person over multiple sessions in order to treat the person for chronic sleep deprivation.

11. The method of claim 3, wherein no surgical or other medical procedure is performed upon the person during performance of the method.

12. A method for treating a person having sleep deprivation, comprising the steps of:
(a) actively inducing a sleep-like state in a person by administering an anesthetic agent to a person;
(b) determining an indication of a stage of sleep of the person by monitoring brainwaves of the person;
(c) maintaining, for a predetermined period of time, the person in a sleep-like state indicated in said step (b) to be deep sleep by adjusting the amount of the anesthetic agent being administered to the person; and
(d) minimizing residual effects of the anesthetic agent in the person upon awakening of the person.

13. The method of claim 12, wherein the anesthetic agent comprises 2-6 diisopropylphenol.

14. The method of claim 12, wherein said step (d) comprises administering a bolus of IV for eliminating accumulation of lactic acid in muscles of the person.

15. The method of claim 14, wherein the bolus of IV fluid comprises a saline solution.

16. The method of claim 12, wherein said step (d) comprises massaging the person for eliminating accumulation of lactic acid in muscles of the person.

17. The method of claim 16, wherein the massaging of the person comprises performing a myofascial massage.

18. The method of claim 12, wherein said step (d) comprises providing food to the person for consumption.

19. The method of claim 12, further comprising providing cognitive behavioral therapy to the person over multiple session in order to treat the person for chronic sleep deprivation.

20. The method of claim 19, wherein no surgical or other medical procedure is performed upon the person during performance of the method.
